(19) United States
(12) Patent Application Publication
(10) Pub. No.: US 2025/0255696 A1
(43) Pub. Date: Aug. 14, 2025

(54) METHODS AND SYSTEMS FOR ANALYZING DENTAL SCANS

(71) Applicant: Align Technology, Inc., San Jose, CA (US)

(72) Inventors: Nikolai ZHIRNOV, Madrid (RU); Vladislav SMIRNOV, Rzhev (RU); Vitalii KOLEVATOV, Kirov (RU); Victor KURTI, Yerevan (AM); Leonid PARAMONOV, Madrid (RU); Grigoriy YAZYKOV, Moscow (RU)

(21) Appl. No.: 19/054,762

(22) Filed: Feb. 14, 2025

Related U.S. Application Data
(60) Provisional application No. 63/553,616, filed on Feb. 14, 2024.

(51) Int. Cl. A61C 7/00; A61C 9/00; A61C 13/34; G06T 19/20; G16H 10/60; G16H 20/40; G16H 30/40

(52) U.S. Cl. CPC A61C 7/002; A61C 9/0053; A61C 13/34; G06T 19/20; G16H 10/60; G16H 20/40; G16H 30/40; G06T 2210/41; G06T 2219/2004

(57) ABSTRACT

Updating and/or optimizing a dental treatment plan can include obtaining a first tooth geometry from a first dental scan. The methods and apparatuses described herein may also include obtaining a second tooth geometry from a second dental scan performed subsequent to the first dental scan. Tooth geometry features from both dental scans may be used to determine corresponding (matching) teeth. Determined positions of the matching teeth can enable treatment plan updating and/or optimizing.

### CLAIM OF PRIORITY

This patent application claims priority to U.S. Provisional Patent Application No. 63/553,616, filed on Feb. 14, 2024, titled “METHODS AND SYSTEMS FOR ANALYZING DENTAL SCANS,” and herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The systems and methods described herein relate generally to analysis of dental scans, and more particularly to the determination of a correspondence between disparate dental scans.

### BACKGROUND

Orthodontic and dental treatments using a series of patient-removable appliances (e.g., “aligners”) are very useful for treating a variety of patients. Treatment planning is typically performed in conjunction with the dental professional (e.g., dentist, orthodontist, dental technician, etc.), by manipulating a model of the patient's teeth from an initial configuration (initial tooth positions) to a final configuration (final tooth positions) and then dividing the treatment into a number of intermediate stages (steps). These steps may correspond to individual appliances that may be worn sequentially, with or without additional interventions (e.g., interproximal reductions, extractions, etc.). Once the treatment plan is finalized, the series of aligners may be manufactured corresponding to the treatment plan.

The treatment plan may begin with a dental scan of the patient's dentition. The dental scan can be the basis of the treatment plan and a three-dimensional model of the patient's teeth may be generated as part of the treatment plan. In some examples, the aligners may be manufactured using information from the three-dimensional model. During the course of treatment by a dental plan, the actual tooth position may not match the treatment plan, or the treatment may be interrupted or delayed partially through the treatment plan. Thus, it would be helpful to provide apparatuses and method for determining how effective a treatment plan is, and/or to adjust, if desired, the remaining stages of the treatment plan.

### SUMMARY OF THE DISCLOSURE

A treatment plan may be optimized part way through the dental treatment. A second dental scan may be performed to determine how effective a treatment plan has been and to help to adjust, if desired, the remaining stages of the treatment plan. In some cases the second dental scan may be performed using different scanning equipment and different scanning operators. As a result, determining a tooth-to-tooth correspondence between the disparate dentals scans can be difficult.

The methods and apparatuses (e.g., devices, system, etc., including software, hardware and/or firmware) described herein may facilitate and improve comparisons between dental scans, e.g., images (including sets of images) and models (e.g., virtual 3D models) of a patient's dentition corresponding to different times. For example, described herein are apparatuses and methods for determining a correspondence between separate dental scans and modifying a dental treatment plan based on the determined correspondence. In general, tooth geometries are determined for teeth in a first (or earlier) dental scan as well teeth in a second (or subsequent) dental scan. By matching the tooth geometries for teeth in the separate dental scans, a correspondence for individual teeth can be determined. Additionally, a geometric mapping can be determined that relates a tooth in the first dental scan to a corresponding tooth in the second dental scan.

In any of the methods described herein, a dental treatment plan may be updated by obtaining a first dental scan based on the dental treatment plan, wherein the dental treatment plan includes a first tooth geometry data and a target position of a patient's teeth, obtaining a second tooth geometry data from a second dental scan performed subsequent to the first dental scan, comparing features in the first tooth geometry data to features in the second tooth geometry data to determine teeth in the first dental scan that correspond to teeth in the second dental scan, determining a geometric transformation to map teeth in the first dental scan that correspond to teeth in the second dental scan, applying the geometric transformation to the patient's teeth in the target position to determine an updated target position, and optimizing one or more stages of the dental treatment plan based on updated target position.

Optimizing dental treatment stages in any of the methods described herein may include applying a constrained optimization adjusting positions of teeth within the one or more stages accounting for changes in teeth shapes between the first dental scan and the second dental scan.

In some examples, optimizing dental treatment stages in any of the methods described herein may include determining that the second dental scan includes at least one newly appearing tooth and modifying one or more stages of the dental treatment plan based at least in part on the at least one newly appearing tooth. Furthermore, the methods may include modifying the one or more stages of the dental treatment plan include adjusting interproximal spaces for teeth in the updated target position based at least in part on the at least one newly appearing tooth.

In any of the methods described herein, optimizing one or more stages of the dental treatment plan may include detecting jaw movement between the target position of the patient's teeth and the second dental scan and modifying one or more stages of the dental treatment plan to accommodate the jaw movement. In some cases, the jaw movement is detected substantially in a sagittal plane of the patient. In some examples, modifying the one or more stages of the dental treatment plan may include limiting tooth movement to a sagittal plane of the patient. In some other examples, modifying the one or more stages of the dental treatment plan may include making inter-arch collisions and anterior-posterior tooth relationships dependent on a relative position of the patient's upper jaw and lower jaw.

In any of the methods described herein, optimizing one or more stages of the dental treatment plan may include determining that the second dental scan includes at least one erupting tooth and modifying one or more stages of the dental treatment plan to accommodate the at least one erupting tooth. In some examples, modifying the one or more stages of the dental treatment plan may include preserving a gap space between teeth in the target position and a gap space between teeth in the one or more modified stages.

In any of the methods described herein, the second dental scan may be provided by an intraoral scanner. In some cases, the second dental scan data can include a three-dimensional (3D) scan of the patient's teeth. In some other cases, the target position can include a 3D model of the patient's teeth in the target position. The 3D model of the patient's teeth can include the first tooth geometry data.

Any of the methods described herein can include generating design files for dental aligners corresponding to optimized stages of the dental treatment plan.

Any of the systems described herein may include one or more processors and a memory configured to store instructions that, when executed by the one or more processors, cause the system to: obtain a dental plan based on a first dental scan, wherein the dental treatment plan includes a first tooth geometry data and a target position of a patient's teeth, obtain a second tooth geometry data from a second dental scan performed subsequent to the first dental scan, compare features in the first tooth geometry data to features in the second tooth geometry data to determine teeth in the first dental scan that correspond to teeth in the second dental scan, determine a geometric transformation to map teeth in the first dental scan that correspond to teeth in the second dental scan, apply the geometric transformation to the patient's teeth in the target position to determine an updated target position, and optimize one or more stages of the dental treatment plan based on updated target position.

In any of the systems disclosed herein, execution of the instructions to optimize one or more stages causes the system to apply a constrained optimization adjusting positions of teeth within the one or more stages accounting for changes in teeth shapes between the first dental scan and the second dental scan.

Further, in any of the systems disclosed herein, execution of the instructions to optimize one or more stages causes the system to: determine that the second dental scan includes at least one newly appearing tooth and modify one or more stages of the dental treatment plan based at least in part on the at least one newly appearing tooth. In some examples, execution of the instructions to modify one or more stages of the dental treatment plan may cause the system to adjust interproximal spaces for teeth in the updated target position based at least in part on the at least one newly appearing tooth.

In any of the systems disclosed herein, execution of the instructions to optimize one or more stages causes the system to detect jaw movement between the target position of the patient's teeth and the second dental scan and modify one or more stages of the dental treatment plan to accommodate the jaw movement. In some examples, the jaw movement is detected substantially in a sagittal plane of the patient. In some cases, in any of the systems disclosed herein, execution of the instructions to modify the one or more stages of the dental treatment plan may cause the system to limit tooth movement to a sagittal plane of the patient. Furthermore, execution of the instructions to modify the one or more stages of the dental treatment plan may cause the system to make inter-arch collisions and anterior-posterior tooth relationships dependent on a relative position of the patient's upper jaw and lower jaw.

In any of the systems disclosed herein, execution of the instructions to optimize one or more stages of the dental treatment plan causes the system to determine the second dental scan includes at least one erupting tooth and modify one or more stages of the dental treatment plan to accommodate the at least one erupting tooth. Furthermore, execution of the instructions to modify the one or more stages of the dental treatment plan can cause the system to preserve a gap space between teeth in the target position and a gap space between teeth in the one or more modified stages.

In any of the systems described herein, the second dental scan may be provided by an intraoral scanner. In any of the systems described herein, the second dental scan data can include a three-dimensional (3D) scan of the patient's teeth. In any of the systems described herein, the target position can include a 3D model of the patient's teeth in the target position. In some cases, the 3D model of the patient's teeth includes the first tooth geometry data.

In any of the systems described herein, execution of the instructions may cause the system to generate design files for dental aligners corresponding to optimized stages of the dental treatment plan.

A non-transitory computer-readable storage medium is disclosed. The non-transitory computer-readable storage medium may include instructions that, when executed by one or more processors of a system, cause the system to perform operations comprising obtaining a dental treatment plan based on a first dental scan, wherein the dental treatment plan includes a first tooth geometry data and a target position of a patient's teeth, obtaining a second tooth geometry data from a second dental scan performed subsequent to the first dental scan, comparing features in the first tooth geometry data to features in the second tooth geometry data to determine teeth in the first dental scan that correspond to teeth in the second dental scan, determining a geometric transformation to map teeth in the first dental scan that correspond to teeth in the second dental scan, applying the geometric transformation to the patient's teeth in the target position to determine an updated target position, and optimizing one or more stages of the dental treatment plan based on updated target position.

Any of the non-transitory computer-readable storage mediums disclosed herein may include instructions to optimize one or more stages of the dental treatment plan that, when executed by one or more processors of a system, cause the system to perform operations comprising applying a constrained optimization adjusting positions of teeth within the one or more stages accounting for changes in teeth shapes between the first dental scan and the second dental scan.

Any of the non-transitory computer-readable storage mediums disclosed herein may include instructions to optimize one or more stages of the dental treatment plan that, when executed by one or more processors of a system, cause the system to perform operations comprising determining that the second dental scan includes at least one newly appearing tooth and modifying one or more stages of the dental treatment plan based at least in part on the at least one newly appearing tooth.

Any of the non-transitory computer-readable storage mediums disclosed herein may include instructions to modify one or more stages of the dental treatment plan that, when executed by one or more processors of a system, cause the system to perform operations comprising adjusting interproximal spaces for teeth in the updated target position based at least in part on the at least one newly appearing tooth.

Any of the non-transitory computer-readable storage mediums disclosed herein may include instructions to optimize one or more stages of the dental treatment plan that, when executed by one or more processors of a system, cause the system to perform operations comprising detecting jaw movement between the target position of the patient's teeth and the second dental scan and modifying one or more stages of the dental treatment plan to accommodate the jaw movement. In some examples, the jaw movement may be detected substantially in a sagittal plane of the patient. In some other examples, execution of the instructions for modifying one or more stages of the dental treatment plan may cause the system to perform operations comprising limiting tooth movement to a sagittal plane of the patient. Furthermore, execution of the instructions for modifying one or more stages of the dental treatment plan may cause the system to perform operations comprising making inter-arch collisions and anterior-posterior tooth relationships dependent on a relative position of the patient's upper jaw and lower jaw.

Any of the non-transitory computer-readable storage mediums disclosed herein may include instructions to optimize one or more stages of the dental treatment plan that, when executed by one or more processors of a system, cause the system to perform operations comprising determining that the second dental scan includes at least one erupting tooth and modifying one or more stages of the dental treatment plan to accommodate the at least one erupting tooth. In some examples, execution of the instructions for modifying one or more stages of the dental treatment plan causes the system to perform operations comprising preserving a gap space between teeth in the target position and a gap space between teeth in the one or more modified stages.

In any of the non-transitory computer-readable storage mediums disclosed herein, the second dental scan is provided by an intraoral scanner. In some examples, the second dental scan data includes a three-dimensional (3D) scan of the patient's teeth. In some other examples, the target position includes a 3D model of the patient's teeth in the target position. The 3D model of the patient's teeth includes the first tooth geometry data.

Any of the non-transitory computer-readable storage mediums disclosed herein may include instructions to modify one or more stages of the dental treatment plan that, when executed by one or more processors of a system, cause the system to perform operations comprising generating design files for dental aligners corresponding to optimized stages of the dental treatment plan.

In any of the methods described herein, a correspondence between teeth within different dental scans can be determined. The method may include obtaining a patient's dental treatment plan based on a first dental scan, wherein the dental treatment plan includes a first tooth geometry data, obtaining a second tooth geometry data from a second dental scan performed subsequent to the first dental scan, and comparing features in the first tooth geometry data to features in the second tooth geometry data to determine teeth in the first dental scan that correspond to teeth in the second dental scan.

In any of the methods described herein, comparing features in the first tooth geometry data to features in the second tooth geometry data include matching tooth buccal ridges of teeth included in the first tooth geometry data to tooth buccal ridges of teeth included in the second tooth geometry data.

In any of the methods described herein, comparing features in the first tooth geometry data to features in the second tooth geometry data include matching tooth tip points of teeth included first tooth geometry data to teeth included second tooth geometry data.

In some examples, in any of the methods described herein, comparing features in the first tooth geometry data to features in the second tooth geometry data may include matching tooth angulation axis of teeth included in the first tooth geometry data to teeth included in the second tooth geometry data.

In some examples, in any of the methods described herein, comparing features in the first tooth geometry data to features in the second tooth geometry data may include matching an occlusal plane of teeth included in the first tooth geometry data to teeth included in the second tooth geometry data.

In some examples, in any of the methods described herein, comparing features in the first tooth geometry data to features in the second tooth geometry data may include matching cusps of teeth included in the first tooth geometry data to teeth included in the second tooth geometry data.

A system is disclosed that may include one or more processors and a memory configured to store instructions that, when executed by the one or more processors, cause the system to obtain a patient's dental treatment plan based on a first dental scan, wherein the dental treatment plan includes a first tooth geometry data, obtain a second tooth geometry data from a second dental scan performed subsequent to the first dental scan, and compare features in the first tooth geometry data to features in the second tooth geometry data to determine teeth in the first dental scan that correspond to teeth in the second dental scan.

In any of the systems disclosed herein, execution of the instructions to compare features in the first tooth geometry data to features in the second tooth geometry data can cause the system to match tooth buccal ridges of teeth included in the first tooth geometry data to tooth buccal ridges of teeth included in the second tooth geometry data.

In any of the systems disclosed herein, execution of the instructions to compare features in the first tooth geometry data to features in the second tooth geometry data can cause the system to match tooth tip points of teeth included first tooth geometry data to teeth included second tooth geometry data.

In any of the systems disclosed herein, execution of the instructions to compare features in the first tooth geometry data to features in the second tooth geometry data can cause the system to match tooth angulation axis of teeth included in the first tooth geometry data to teeth included in the second tooth geometry data.

In any of the systems disclosed herein, execution of the instructions to compare features in the first tooth geometry data to features in the second tooth geometry data can cause the system to match an occlusal plane of teeth included in the first tooth geometry data to teeth included in the second tooth geometry data.

In any of the systems disclosed herein, execution of the instructions to compare features in the first tooth geometry data to features in the second tooth geometry data can cause the system to match cusps of teeth included in the first tooth geometry data to teeth included in the second tooth geometry data.

A non-transitory computer-readable storage medium is disclosed. The non-transitory computer-readable storage medium may include instructions that, when executed by one or more processors of a system, cause the system to perform operations comprising obtaining a patient's dental treatment plan based on a first dental scan, wherein the dental treatment plan includes a first tooth geometry data, obtaining a second tooth geometry data from a second dental scan performed subsequent to the first dental scan, and comparing features in the first tooth geometry data to features in the second tooth geometry data to determine teeth in the first dental scan that correspond to teeth in the second dental scan.

In any of the non-transitory computer-readable storage mediums disclosed herein, execution of the instructions for comparing features in the first tooth geometry data to features in the second tooth geometry data may cause the system to perform operations comprising matching tooth buccal ridges of teeth included in the first tooth geometry data to tooth buccal ridges of teeth included in the second tooth geometry data.

In any of the non-transitory computer-readable storage mediums disclosed herein, execution of the instructions for comparing features in the first tooth geometry data to features in the second tooth geometry data may cause the system to perform operations comprising matching tooth tip points of teeth included first tooth geometry data to teeth included second tooth geometry data.

In any of the non-transitory computer-readable storage mediums disclosed herein, execution of the instructions for comparing features in the first tooth geometry data to features in the second tooth geometry data may cause the system to perform operations comprising include matching tooth angulation axis of teeth included in the first tooth geometry data to teeth included in the second tooth geometry data.

In any of the non-transitory computer-readable mediums disclosed herein, execution of the instructions for comparing features in the first tooth geometry data to features in the second tooth geometry data may cause the system to perform operations comprising include matching an occlusal plane of teeth included in the first tooth geometry data to teeth included in the second tooth geometry data.

In any of the non-transitory computer-readable mediums disclosed herein, execution of the instructions for comparing features in the first tooth geometry data to features in the second tooth geometry data causes the system to perform operations comprising include matching cusps of teeth included in the first tooth geometry data to teeth included in the second tooth geometry data.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 is a diagram illustrating one variation of a dental computing environment.

FIG. 2 is a block diagram of one example of treatment monitoring modules.

FIGS. 3A and 3B show different example scans of a patient's upper arch.

FIGS. 4A and 4B show different scanned images of the same molar tooth.

FIGS. 5A and 5B show different scanned images of the same tooth.

FIG. 6 is a flowchart showing an example method for optimizing a patient's treatment plan.

FIG. 7 shows an image depicting example tooth geometries that may be used to determine matching teeth.

FIG. 8 shows another image depicting other example tooth geometries that may be used to determine matching teeth.

FIG. 9 shows another image depicting other example tooth geometries that may be used to determine matching teeth.

FIG. 10 shows an image of an upper arch.

FIGS. 11A and 11B illustrate the matching of features of teeth between different dental scans.

FIG. 12 depicts teeth with improved leveling as a result of optimized treatment planning.

FIG. 13 depicts teeth with decreased X-misalignment issues as a result of optimized treatment planning.

FIG. 14 is a flowchart showing another example method for optimizing a patient's treatment plan.

FIG. 15 is a flowchart showing an example method for accommodating newly appearing teeth.

FIG. 16 shows an example 3D model.

FIG. 17 is a flowchart showing an example method for accommodating movement in a horizontal (sagittal) plane.

FIG. 18 shows an example user interface to optimize tooth position while allowing only sagittal plane movement.

FIG. 19 shows an example user interface for lower jaw movement visualization.

FIG. 20 is a flowchart showing an example method for accommodating erupting teeth.

FIG. 21A shows a first view of a partial dental arch.

FIG. 21B shows a second view of the partial dental arch of FIG. 21A.

FIG. 22A shows a non-fully erupted tooth in a current scan.

FIG. 22B shows a primary (or deciduous) tooth that may be associated with an earlier scan.

FIG. 23A shows a permanent tooth erupting into position in a subsequent scan.

FIG. 23B shows a primary tooth in position during an earlier scan.

### DETAILED DESCRIPTION

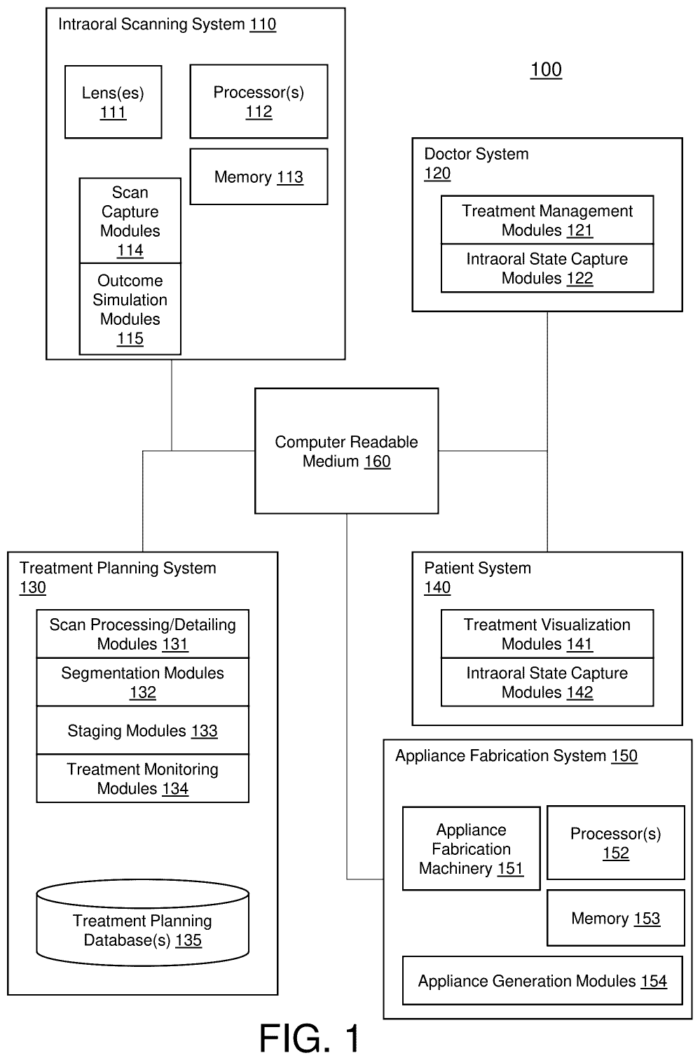

In general, the methods and apparatuses described herein may assist in comparing teeth between different scans of a patient's dentition, and in particular may be useful to help modify or confirm a treatment plan (e.g., a new treatment plan, or a preexisting treatment plan). Any of these methods and apparatuses may be used at one or more parts of a dental computing environment, including as part of an intraoral scanning system, doctor system, treatment planning system, patient system, and/or fabrication system. In particular, these methods and apparatuses may be used as part of a treatment planning system, for example, to determine an accurate (in some cases initial) location of a patient's teeth. The initial location may be used to determine a final location for one or more of the patient's teeth based on a treatment plan. The treatment plan may be optimized (modified) based on updated dental scans. For example, FIG. 1 is a diagram illustrating one variation of a dental computing environment 100 that may generate one or more orthodontic treatment plans specific to a patient, and fabricate dental appliances that may accomplish the treatment plan to treat a patient, under the direction of a dental professional. The example dental computing environment 100 shown in FIG. 1 includes an intraoral scanning system 110, a doctor system 120, a treatment planning system 130, a patient system 140, an appliance fabrication system 150, and computer-readable medium 160. In some variations the dental computing environment (sometimes referred to as a dental computing system) 100 may include just one or a subset of these systems (which may also be referred to as sub-systems of the overall system 100). Further, one or more of these systems may be combined or integrated with one or more of the other systems (sub-systems), such as, e.g., the treatment planning system 130 and the doctor system 120 may be part of a remote server accessible by a doctor interface. The computer readable medium 160 may divided between all or some of the systems (subsystems); for example, the treatment planning system 130 and the appliance fabrication system 150 may be part of the same sub-system and may be on a computer readable medium 160. Further, each of these systems may be further divided into sub-systems or components that may be physically distributed (e.g., between local and remote processors, etc.) or may be integrated.

An intraoral scanning system may include an intraoral scanner as well as one or more processors for processing images. For example, the intraoral scanning system 110 can include lens(es) 111, processor(s) 112, a memory 113, scan capture modules 114, and outcome simulation modules 115. In general, the intraoral scanning system 110 can capture one or more images of a patient's dentition. Use of the intraoral scanning system 110 may be in a clinical setting (doctor's office or the like) or in a patient-selected setting (the patient's home, for example). In some cases, operations of the intraoral scanning system 110 may be performed by an intraoral scanner, dental camera, cell phone or any other feasible device.

The lens(es) 111 include one or more lenses and optical sensors to capture reflected light, particularly from a patient's dentition. The scan capture modules 114 can include instructions (such as non-transitory computer-readable instructions) that may be stored in the memory 113 and executed by the processor(s) 112 to control the capture of any number of images of the patient's dentition.

As mentioned, in some examples the methods and apparatuses described herein for generating a 3D model including one or more teeth may be part of, or accessible by, the intraoral scanning system 110, computer readable medium 160 and/or treatment planning system 130.

For example, the outcome simulation modules 115, which may be part of the intraoral scanning system 110, can include instructions that simulate final tooth positions based on a treatment plan. In some cases, the outcome simulation modules 115 can include instructions that simulate tooth positions using images or other scan data from a dental scan

Any of the component systems or sub-systems of the dental computing environment 100 may access or use the patient's dental information including scan data, three-dimensional (3D) dental models, and/or treatment plans generated by the methods and apparatuses described herein. For example, the doctor system 120 may include treatment management modules 121 and intraoral state capture modules 122 that may access or use patient scan data and/or 3D dental models. The doctor system 120 may provide a “doctor facing” interface to the computing environment 100. The treatment management modules 121 can perform any operations that enable a doctor or other clinician to manage the treatment of any patient. In some examples, the treatment management modules 121 may provide a visualization and/or simulation of the patient's dentition with respect to a treatment plan. For example, the doctor system 120 may include a user interface for the doctor that allows the doctor to manipulate and view a patient's 3D dental model.

The intraoral state capture modules 122 can provide images of the patient's dentition to a clinician through the doctor system 120. The images may be captured through the intraoral scanning system 110 and may also include images of a simulation of tooth movement based on a treatment plan.

In some examples, the treatment management modules 121 can enable the doctor to modify or revise a treatment plan. The doctor system 120 may include one or more processors configured to execute any feasible non-transitory computer-readable instructions to perform any feasible operations described herein.

Alternatively or additionally, the treatment planning system 130 may include any of the methods and apparatuses described herein, and/or may determine a mapping between dental scans of a patient that are displaced in time. The treatment planning system 130 may include scan processing/detailing modules 131, segmentation modules 132, staging modules 133, treatment monitoring modules 134, treatment planning database(s) 135, and ridgeline management modules 136. In general, the treatment planning system 130 can determine a treatment plan for any feasible patient. The scan processing/detailing modules 131 can receive or obtain dental scans (such as scans from the intraoral scanning system 110) and can process the scans to “clean” them by removing scan errors and, in some cases, enhancing details of the scanned image.

The treatment planning system 130 may include a segmentation system that segments a model into separate components. For example, the treatment planning system 130 may include a segmentation modules 132 that can segment a dental model (such as a 3D dental model) into separate parts including separate teeth, gums, jaw bones, and the like. In some cases, the dental models may be based on scan data from the scan processing/detailing modules 131.

The staging modules 133 may determine different stages of a treatment plan. Each stage may correspond to a different dental aligner. In some examples, the staging modules 133 may also determine the final position (also referred to as target position) of the patient's teeth, in accordance with a treatment plan. Thus, the staging modules 133 can determine some or all of a patient's orthodontic treatment plan. In some examples, the staging modules 133 can simulate movement of a patient's teeth in accordance with the different stages of the patient's treatment plan.

The treatment monitoring modules 134 can monitor the progress of an orthodontic treatment plan. In some examples, the treatment monitoring modules 134 can provide an analysis of progress of treatment plans to a clinician. The orthodontic treatment plans may be stored in the treatment planning database(s) 135. Although not shown here, the treatment planning system 130 can include one or more processors configured to execute any feasible non-transitory computer-readable instructions to perform any feasible operations described herein.

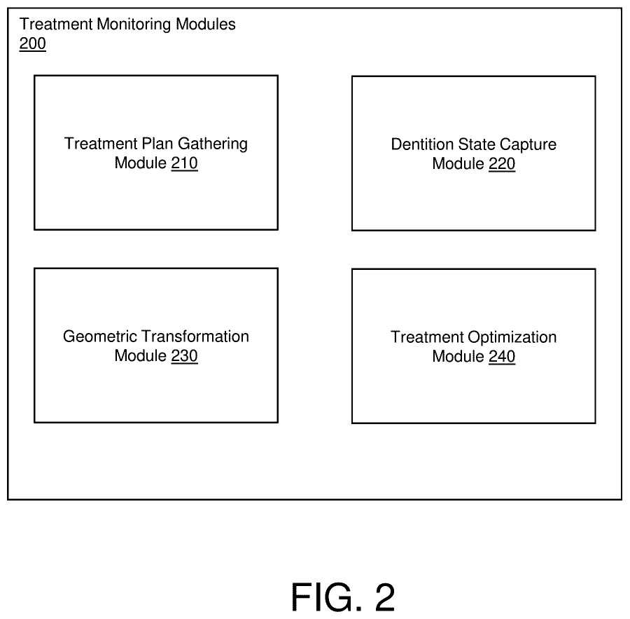

In some examples, the treatment monitoring modules 134 may further include modules that can determine the relationship between a patient's teeth that are included in two separate dental scans. For example, the treatment monitoring modules 134 can determine a geometric relationship between teeth that are associated with a first dental scan and teeth that are associated with a second dental scan, where the first and second dental scans are separated by time. In some cases the time may be one month, several months, several years, or the like. Further details of the treatment monitoring modules 134 are described herein with respect to FIG. 2. Notably, the functionality of determining a relationship between teeth included in separate dental scans is not limited to the treatment planning system 130, but can be included within any other portion of the dental computing environment 100.

The patient system 140 can include treatment visualization modules 141 and intraoral state capture modules 142. In general, the patient system 140 can provide a “patient facing” interface to the computing environment 100. The treatment visualization modules 141 can enable the patient to visualize how an orthodontic treatment plan has progressed and also visualize a predicted outcome (e.g., a final position of teeth).

In some examples, the patient system 140 can capture dentition scans for the treatment visualization modules 141 through the intraoral state capture modules 142. The intraoral state capture modules 142 can enable a patient to capture his or her own dentition through the intraoral scanning system 110. Although not shown here, the patient system 140 can include one or more processors configured to execute any feasible non-transitory computer-readable instructions to perform any feasible operations described herein.

The appliance fabrication system 150 can include appliance fabrication machinery 151, processor(s) 152, memory 153, and appliance generation modules 154. In general, the appliance fabrication system 150 can directly or indirectly fabricate aligners to implement a dental treatment plan. In some examples, the dental treatment plan may be stored in the treatment planning database(s) 135.

The appliance fabrication machinery 151 may include any feasible implement or apparatus that can fabricate any suitable dental aligner. The appliance generation modules 154 may include any non-transitory computer-readable instructions that, when executed by the processor(s) 152, can generate one or more design files that can correspond to stages determined by the staging modules 133. In turn, the one or more design files may be used to build or fabricate one or more dental aligners. In some examples, the appliance fabrication machinery 151 can use the design files to produce the one or more dental aligners. The memory 153 may store data or instructions for use by the processor(s) 152. In some examples, the memory 153 may temporarily store a treatment plan, dental models, or intraoral scans.

The computer-readable medium 160 (sometimes referred to as a non-transitory computer-readable storage medium) may include some or all of the elements described herein with respect to the dental computing environment 100. The computer-readable medium 160 may include non-transitory computer-readable instructions that, when executed by a processor, can provide the functionality of any device, machine, or module described herein.

FIG. 2 is a block diagram of one example of treatment monitoring modules 200. The treatment monitoring modules 200 may be an example of the treatment monitoring modules 134 of FIG. 1. The treatment monitoring modules 200 may include a treatment plan gathering module 210, a dentition state capture module 220, a geometric transformation module 230, and a treatment optimization module 240. In general, the treatment monitoring system 200 can optimize or update a patient's treatment plan based on an updated dental scan. The updated dental scan may include tooth geometry information and also recent tooth position information. In some examples, a patient's existing treatment plan may be modified based on the updated dental scan.

The treatment plan gathering module 210 can retrieve a patient's treatment plan. In some cases, the patient's treatment plan may be stored in, and retrieved from, the treatment planning database(s) 135 of FIG. 1. In some examples, the patient's treatment plan may include the patient's tooth geometry information and a 3D model of the patient's dentition. The treatment plan may be used to move the patient's teeth into a target position. Thus, the patient's treatment plan may be implemented with one or more stages, where each stage may be associated with a dental aligner.

The dentition state capture module 220 can capture or obtain any feasible images of the patient's dentition. In some examples, the dentition state capture module 220 can execute or perform any operations described with respect to the intraoral scanning system 110. Thus, the dentition state capture module 220 can capture an updated dental or oral scan of the patient that is subsequent to a dental or oral scan used to determine the dental treatment plan.

The geometric transformation module 230 can determine a relationship between teeth that are included in a final position (target position) of a treatment plan and teeth that are included in a subsequent dental scan. In particular, the subsequent dental scan may be a second dental scan that has been captured after a first dental scan, where the first dental scan was used in formulating the treatment plan. In some examples, the geometric transformation module 230 can determine a mathematic or geometric relationship between the teeth in a target position (associated with the treatment plan) and the teeth in an updated dental scan. In many cases, the geometric relationship can describe how the two teeth are related between different dental scans, different dental data sets, different dental 3D models, and the like.

In some examples, information from the geometric transformation module 230 may be used to identify like, similar, or corresponding teeth. For example, the same tooth may appear to be different teeth on separate scans. The difference may be due to small tooth changes, a small positional change, or the like. The small differences may cause the teeth to appear as different teeth, particularly when the teeth are compared by a processor executing a software-based comparison routine.

The geometric transformation module 230 can be used to identify corresponding (related) teeth that are in separate dental scans. After the teeth are identified, then the patient's treatment plan can be optimized.

The treatment optimization module 240 may be used to optimize or otherwise adjust a treatment plan based on information from a dental scan. For example, a patient's treatment plan can be determined using a first or initial dental scan. The treatment plan can include multiple stages. Each stage can be associated with a dental aligner that is used to reposition the patient's teeth to achieve a target position (final position).

The treatment optimization module 240 can access the original treatment plan, in particular to review the final or target position of the patient's teeth. Then, using the location of the patient's current teeth, the treatment optimization module 240 can adjust or revise the patient's treatment plan if necessary to achieve the final or target position of the original treatment plan. The revised treatment plan may include revised or new stages. In turn, the revised or new stages may require new dental aligners.

The embodiments described herein may be used to monitor and optimize a patient's dental treatment plan. While a first or initial dental scan may be used to determine the patient's treatment plan, a second dental scan may be performed at any time during the course of treatment. The scan data from the second dental scan may be used to modify or optimize the patient's treatment plan.

In order to provide the most accurate optimization, a correspondence between teeth is found in the first and second dental scans. In some cases, a geometric transformation may be used to describe the relationship between teeth in a first dental scan to teeth in a second dental scan. After the relationship has been established, tooth position from the second dental scan may be used to modify/optimize the patient's treatment.

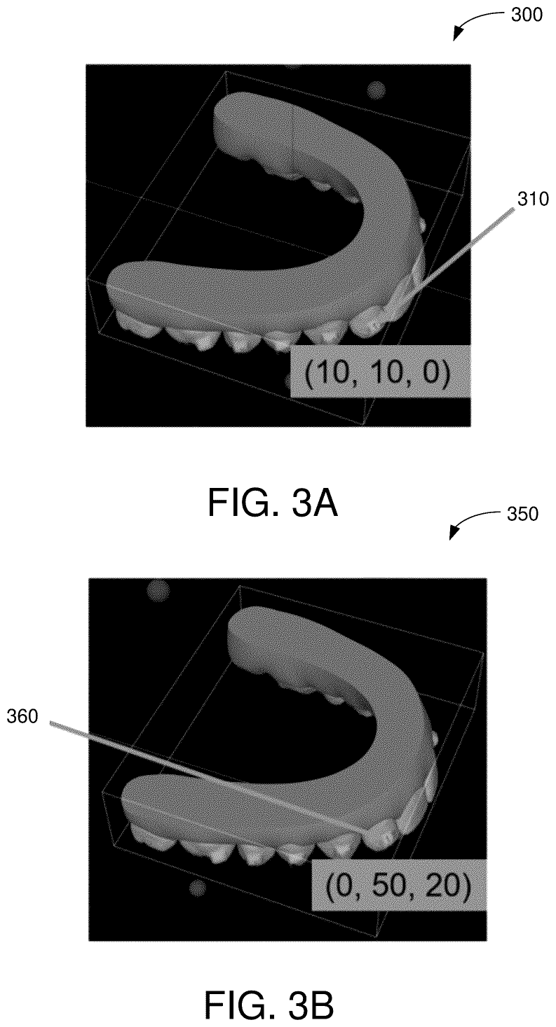

FIGS. 3A and 3B show different example scans of a patient's upper arch. The upper arch 300 of FIG. 3A may be associated with a first dental scan and the upper arch 350 of FIG. 3B may be associated with a second dental scan. The two dental scans may be associated with different scanning times. For example, the upper arch 300 may be associated with a first dental scan and the upper arch 350 may be associated with a second or subsequent dental scan that has taken place after the first dental scan. In some aspects, the second dental scan may be associated with a scan taken after a treatment plan has begun.

Because the dental scans have been taken at two separate times, the 3D coordinates of features or characteristics of the teeth may be subject to slightly different coordinate systems, or similar coordinate systems with different origins. In addition, in some cases a tooth may have moved either in position (e.g., x, y, z) or rotation (e.g., pitch, yaw, roll or θ, ϕ, ψ). In either case, any of these circumstances can result in causing a tooth not to be recognized as the same tooth in both scans.

As an example, a feature 310 on a tooth in the first upper arch 300 can have coordinates (10, 10, 0). Same feature 360 on the same tooth in the second upper arch 350 can have coordinates (0, 50, 20). Thus, in some cases, one or more teeth in different scans may appear as different teeth instead of appearing correctly as the same tooth.

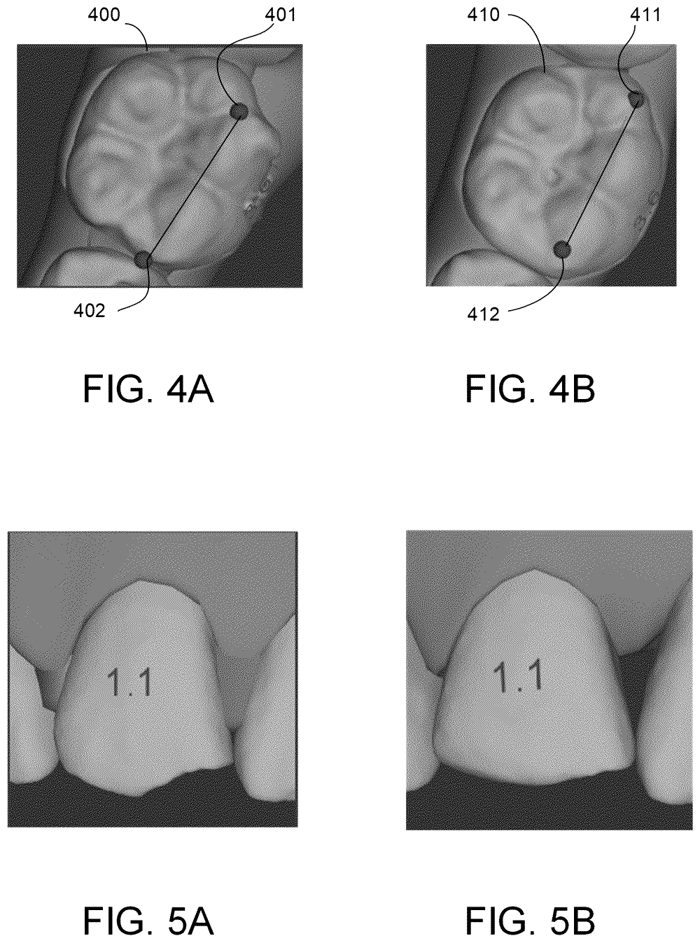

As a further example, FIGS. 4A and 4B show different scanned images of the same molar tooth. The molar 400 in FIG. 4A may be associated with a first dental scan and the molar 410 in FIG. 4B may be associated with a second or subsequent dental scan. In some cases, the same tooth may appear different due to different 3D dental scanners and/or different computer aided design (CAD) operators. The molar 400 shows a buccal ridge defined by points 401 and 402. The molar 410 shows a buccal ridge defined by points 411 and 412. Although the tooth is the same in FIGS. 4A and 4B, the different buccal ridges may cause the tooth to be interpreted as different teeth.

In another example, FIGS. 5A and 5B show different scanned images of the same tooth. The tooth 500 in FIG. 5A may be associated with a first dental scan and the tooth 510 in FIG. 5B may be associated with a second or subsequent dental scan. In some examples, tooth shape in a second scan may differ from the tooth shape in the first scan due to physical tooth damage or restoration. Notably, the shape of tooth 500 may differ from the tooth 510. The different shape may cause the tooth 500 to be interpreted as a different tooth with respect to tooth 510.

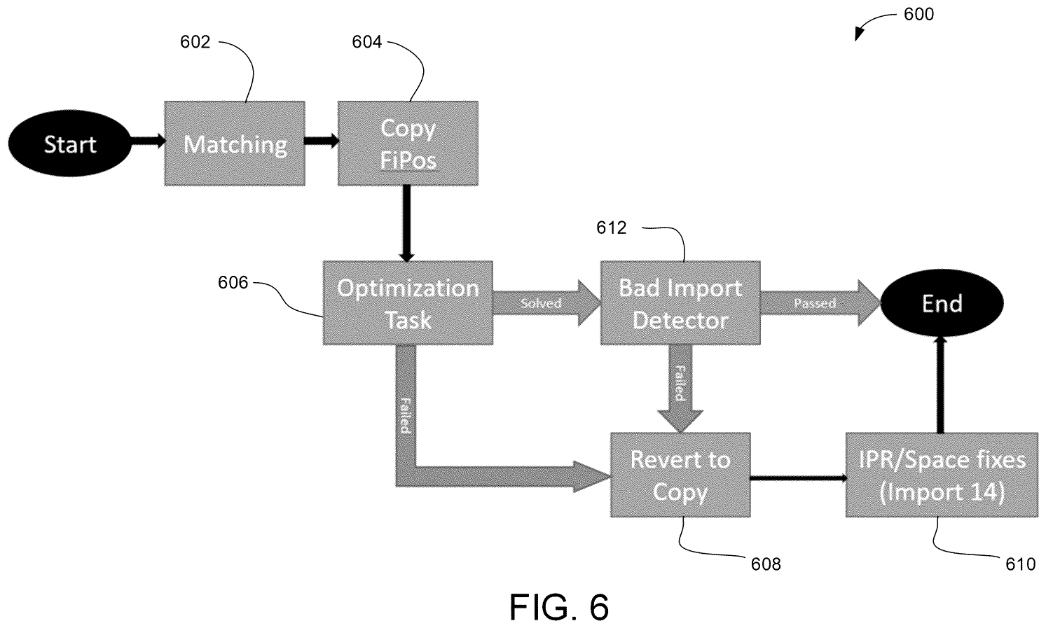

FIG. 6 is a flowchart showing an example method 600 for optimizing a patient's treatment plan. Some examples may perform the operations described herein with additional operations, fewer operations, operations in a different order, operations in parallel, and some operations differently. In general, the method 600 may be used to adjust or update a treatment plan, particularly after data from a second dental scan has become available. The method 600 is just one example of how a patient's treatment plan may be optimized. Other methods to optimize a treatment plan using a second or updated dental scan may exist.

The method 600 begins in block 602 where teeth associated with a second (later) dental scan are matched with corresponding teeth that are associated with a first (earlier) dental scan. The matching may include determining or identifying one or more characteristics on each tooth. The characteristics may be any feature (in some cases, external tooth geometries) that may help identify a tooth. For example, tooth features such as buccal ridges, cusps, tip points (tips appearing on or near a biting surface), angulation axis, occlusal planes, or the like may be used to identify teeth.

In some cases, the first dental scan may be provided or included with a patient's dental treatment plan. The patient's dental treatment plan may be used to move the patient's teeth from an initial or starting position to target or final position. The movement is typically broken into separate stages. A separate dental aligner may be associated with each stage. Thus, different dental aligners may be worn by the patient to implement different stages of the dental treatment plan.

Since the first dental scan and the second dental scan typically are independent events, the coordinate system used to describe the patient's teeth may differ between the first dental scan and the second dental scan. In some cases, even using the same scanning equipment, the coordinate system may change due to environmental conditions or due to different scanning technicians. The coordinate system is used to describe any physical attributes of the patient's teeth. The different coordinate systems may make computer assisted recognition of the patient's teeth and treatment optimization difficult.

Therefore, in block 602 a transformation (such as a geometric transformation) may be determined that describes how a matching tooth in the first dental scan corresponds to a tooth in the second dental scan. Note that the transformation for each matching tooth may differ from any other matching tooth. In some examples, the transformation may describe how the matching teeth can relate to each other through a transformation equation that can relate the teeth together through a movement or translation in position (e.g., x, y, z) or rotation (e.g., pitch, yaw, roll or θ, ϕ, ψ). Although described herein with six degrees of freedom, in other examples other transformations may be used.

Next, in block 604 a target position (final position) may be obtained or “copied” from the patient's dental treatment plan. The target position can describe the desired final position of the patient's teeth as determined by a clinician.

Next, in block 606 the patient's treatment plan may be optimized based on the second dental scan. The second dental scan can provide a “current” position of the patient's teeth. The target position from the patient's original treatment plan can be used with the current position of the patient's teeth to determine if one or more stages of the patient's treatment plan can or should be modified. The matching of block 602 enables the identification of teeth in the second dental scan that correspond to teeth in the target position of the treatment plan.

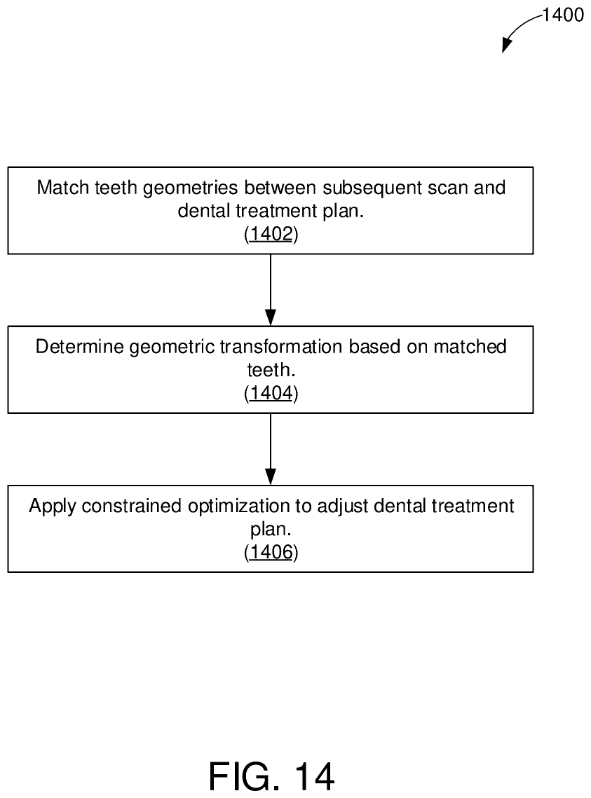

After identifying the teeth in the second dental scan that correspond or match teeth in the target position of the treatment plan, the stages for the treatment plan may be optimized or updated. Updating the treatment plan may include preserving or modifying interproximal spaces, modifying tooth movement or the like. Updating the treatment plan is described in more detail with respect to FIGS. 14-23.

The optimization task for block 606 may either be solved (successful) or failed (unsuccessful). A successful optimization is one where a positive correspondence between teeth that are in the second dental scan is found to teeth in the first dental scan. In some examples, a successful optimization occurs then all the teeth in the second dental scan have a corresponding tooth in the first dental scan. A failed optimization, therefore, may be one where a positive correspondence between teeth in the second dental scan is not found to teeth in the first dental scan. When no correspondence is found, then the patient's dental plan cannot be optimized.

In the case of an optimization failure, the method can proceed to block 608 where the original dental treatment plan is used. The method proceeds to block 610 where interproximal spaces are fixed. The IPR fix may be a simple movement of teeth along the arch to achieve the desired inter-proximal space/contact relationship. Any improvement on position may be implemented as part of the fix, typically to improve position of teeth after the CopyFiPos step 604. Next, the method may end.

Returning to block 606, if the optimization was successful, then in block 612 a check for a bad import. Checking for a bad import may provide a quality check procedure that does not require implementing an optimization task which controls all positional parameters of a tooth/jaws (e.g., 3 rotations and 3 translations for each tooth and jaw-jaw position); instead this technique may add a control one by one. In some cases insufficient control during the optimization task may result in a final position of poor quality. The bad import check may decrease the number of tests performed by bad import detector during development of the optimization task, increasing applicability of optimization engine.

If no bad import is detected, then the method 600 can end. On the other hand, if a bad import is detected, then the method proceeds to block 608.

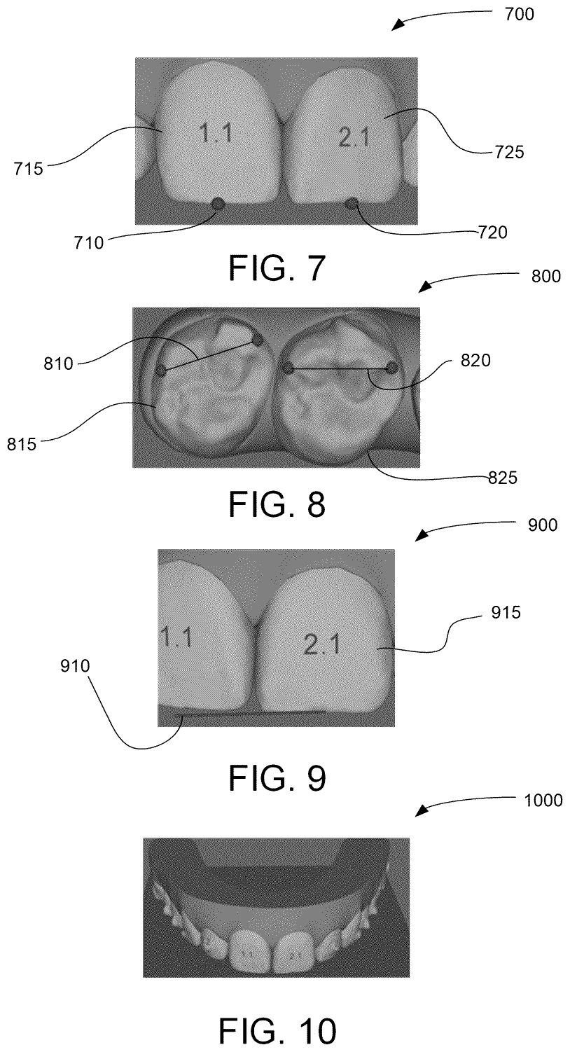

FIG. 7 shows an image 700 depicting example tooth geometries that may be used to determine matching teeth. For example, image 700 include a first tooth tip point 710 on a first tooth 715 and a second tooth tip point 720 on a second tooth 725. The first tooth tip point 710 and the second tooth tip point 720 may be used to identify the first tooth 715 and the second tooth 725, respectively in different dental scans. In general, any tooth geometries or features may be used to identify matching teeth. In some examples, more than one tooth geometry or feature is used to identify matching teeth.

FIG. 8 shows another image 800 depicting other example tooth geometries that may be used to determine matching teeth. For example, image 800 shows a first buccal ridge 810 on a first tooth 815 and a second buccal ridge 820 on a second tooth 825. The first buccal ridge 810 and the second buccal ridge 820 may be used to identify the first tooth 815 and the second tooth 825, respectively in separate dental scans.

However in some cases the tooth/jaw feature calculation may be unstable (e.g., for tooth geometries with minor changes, tooth feature position may change significantly) and may not be used to define tooth geometry matching. Thus, the methods and apparatuses described herein may, in some cases, use only tooth geometries for matching. Tooth geometry matching may be used to define matching of tooth/jaw features. Tooth features may be used to formulate the parameters which may be copied from a previous order. The information on tooth/jaw feature matching may be used to correctly measure the copied values on the previous order, since the previous order may give the exact deviation between the tooth/jaw feature in the new/previous order. The deviation may be caused by instability in tooth/jaw feature calculation or different flows, or physically different geometries as described above. For example, if the method or apparatus copies leveling which, in a previous order may be 2 mm; tooth feature matching may give the information that tip point deviation between new/previous orders is 1 mm. Having this information in the new order may achieve 1 mm leveling (e.g., 2 mm from the previous order, resulting in 1 mm of tooth feature matching discrepancy).

FIG. 9 shows another image 900 depicting other example tooth geometries that may be used to determine matching teeth. For example image 900 shows an occlusal plane 910 of a tooth 915. In some examples, an occlusal plane is a flat plane that passes near incisal edges of the incisors and the tip of the occluding surfaces of the posterior teeth. Thus, the occlusal plane 910 may be used to identify the tooth 915 in separate dental scans.

In general, to perform treatment plan optimization (as described with respect to block 606 of FIG. 6), the position of the patient's teeth in a current scan needs to be associated with the position of the patient's teeth in an earlier scan. In some cases, treatment plan optimization can include determining (or developing, modifying) a treatment plan to move the patient's teeth from position indicated in a current scan to the target position of a preexisting treatment plan. In some examples, a geometric transformation may be applied to teeth in an earlier dental scan (for example, in a target position) to teeth in a current scan to determine updated stages of a dental treatment plan. FIG. 10 shows an image of an upper arch 1000 that may include any number of tooth geometries that may be used to identify any particular teeth.

Generally, optimizing a treatment plan can begin with calculating or determining tooth geometries (features) for teeth that are in a dental scan. Often, this dental scan is associated with a subsequent dental scan (that is, a dental scan generally performed after treatment has started). As described above, the tooth geometries can be any features that can assist in determining or identifying a tooth. Next, the tooth geometries may be determined associated with another dental scan. Often, this dental scan may be associated with an original (initial) dental treatment plan.

Next, teeth within the different dental scans are matched, using the tooth geometries described above. In this manner, the teeth that are the same in both scans can be identified and matched between the scans. Then, using the target (final) position of the original dental treatment plan, upcoming treatment stages can be optimized or modified based on the current position of the teeth.

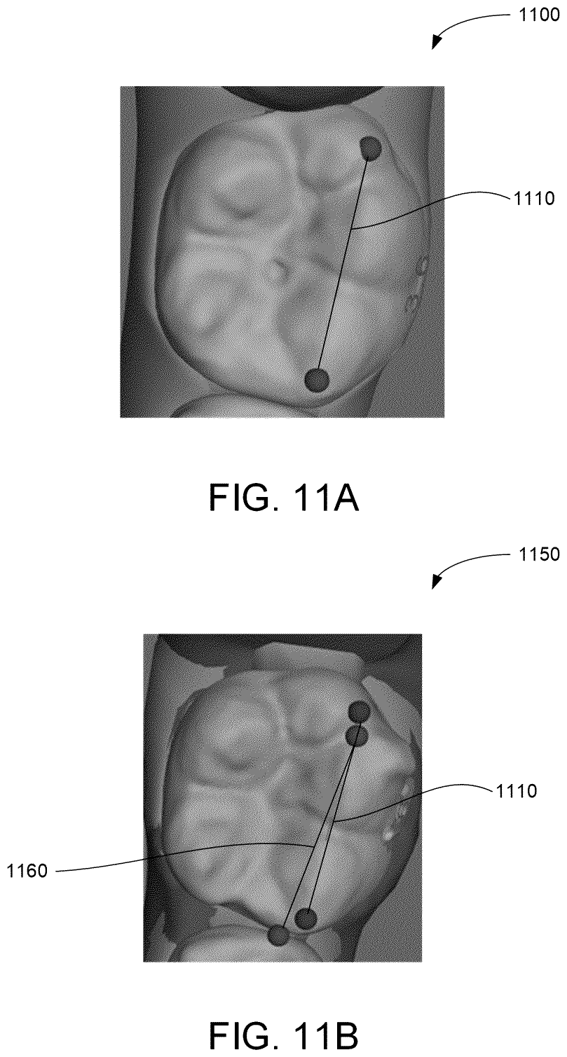

FIGS. 11A and 11B illustrate the matching of features of teeth between different dental scans. FIG. 11A shows a tooth 1100 associated with a subsequent dental scan. A ridge 1110 associated with tooth 1100 has been identified. FIG. 11B shows a tooth 1150 associated with a dental treatment plan. A ridge 1160 may also be associated with the tooth 1150. In FIG. 11B, the ridge 1110 has been superimposed over the tooth 1150. The difference between the ridge 1110 and the ridge 1160 may be due to a different scanner, a different scanner operator (clinician), or some other difference which can manifest as small differences between scans.

After matching teeth between the different dental scans, the dental treatment plan can be optimized based on the subsequent dental scan. In this manner, any changes or adjustments based on detected tooth movement can be made.

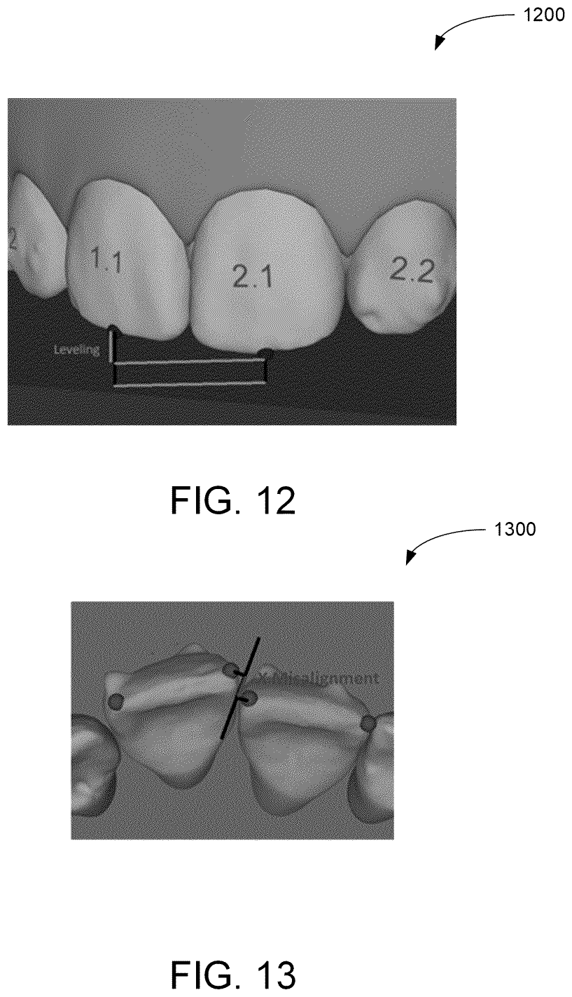

FIG. 12 depicts teeth 1200 with improved leveling as a result of optimized treatment planning using the tooth geometry (tooth features) matching described herein. Leveling describes a relative position of a pair of teeth with respect to intrusion/extrusion directions. Leveling can be calculated as a distance between tip points (the farthest point of the tooth along the long axis that lies on the incisal edge) in millimeters. In some cases, leveling issues may be decreased from 20% to 1% using the methods described herein.

FIG. 13 depicts teeth 1300 with decreased X-misalignment issues as a result of optimized treatment planning. X-misalignment describes a relative position of a pair of teeth in a buccal/lingual direction. X-misalignment may be calculated as a distance between the closest edges of tooth ridges in millimeters. In some cases, X-misalignment issues may be decreased from 12.5% to 1.5%.

FIG. 14 is a flowchart showing another example method 1400 for optimizing a patient's treatment plan. Some examples may perform the operations described herein with additional operations, fewer operations, operations in a different order, operations in parallel, and some operations differently. In general, the method 1400 may be wholly or partially within the method 600 of FIG. 6.

The method 1400 may begin in block 1402 where tooth geometries are matched between tooth information captured or included within a dental treatment plan and tooth geometries included within a dental scan, such as a subsequent dental scan. A subsequent dental scan may be a dental scan performed any time after an initial dental scan used to determine the dental treatment plan.

The matching of block 1402 can include determining tooth geometries associated with a dental treatment plan. For example, a 3D model of the patient's teeth in a final position (target position) may be used to determine a set of tooth geometries. The matching of block 1402 can also include determining tooth geometries associated with teeth in a dental scan, such as a dental scan performed after a dental treatment has begun.

Next, in block 1404, a geometric relationship (transformation) is determined based at least in part on the matched teeth of block 1402. In some examples, the geometric transformation can include determining how to relate a first tooth in a first scan to the same tooth in a second scan. In some cases, matched teeth may be described with by a relationship showing how the teeth may be related through six degrees of freedom (e.g., x, y, z, and pitch, yaw, roll or θ, ϕ, ψ).

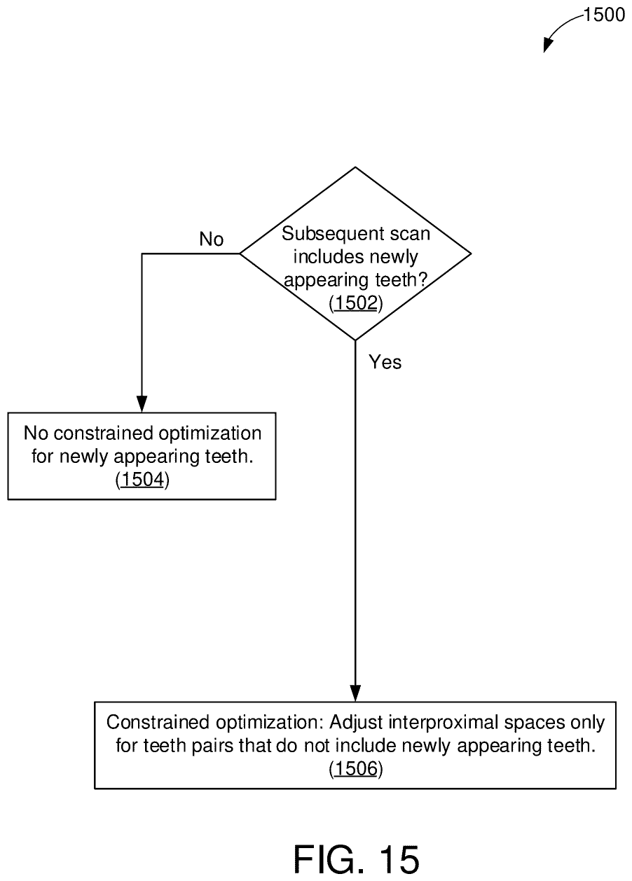

Next, in block 1406, a constrained optimization may be applied to adjust a patient's dental treatment plan. In some examples, the constrained optimization can be used to accommodate newly appearing teeth, detected jaw movement, and non-fully erupted teeth. These optimizations are described below in conjunction with FIGS. 15-23.

FIG. 15 is a flowchart showing an example method 1500 for accommodating newly appearing teeth. In general, the method 1500 may be used to adjust a dental treatment plan so that newly appearing teeth may safely erupt without undue collisions or impacts due to existing teeth. The method 1500 may be wholly or partially included with the method 1400 of FIG. 14, or in some cases wholly or partially included within block 606 of FIG. 6.

The method 1500 may begin in block 1502 where the new or subsequent dental scan is examined for newly appearing teeth. A benefit of the matching (block 1402 of FIG. 14) is that newly appearing teeth may be easily identified in a new or subsequent dental scan. For example, any teeth that do not match between an earlier dental scan (or scan data, such as a target or final position data) may be due to a new tooth.

If no new teeth are found, then no constrained optimization for newly appearing teeth is required and the method 1500 may end. On the other hand, if newly appearing teeth are detected, then in block 1506 the patient's dental plan may undergo constrained optimization. For example, interproximal spaces may only be adjusted for teeth pairs that do not include newly appearing teeth. In this manner, newly appearing teeth are allowed to have space or room to fully erupt and grow into the patient's dental arch.

Generally, optimization applies normal interproximal contacts between neighboring teeth. However, if there was a space between these teeth in a target position (e.g., an initial final position), constrained optimization maintains or keeps this space. If there was an IPR (Interproximal Reduction) between these teeth in the initial dental treatment plan, then constrained optimization checks the position of adjacent teeth to determine if the tooth-to-tooth contact exists and whether an IPR can be created.

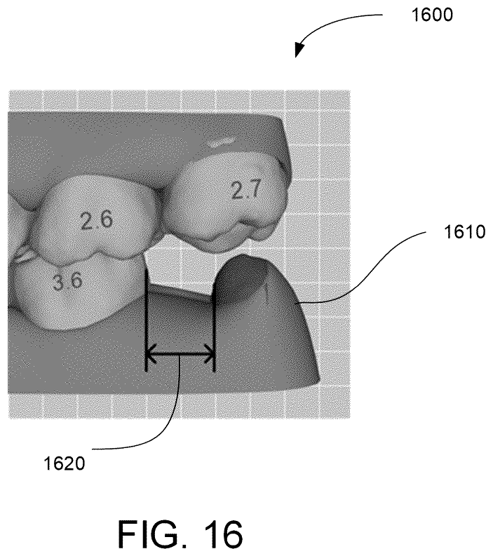

FIG. 16 shows an example 3D model 1600. A newly appearing tooth 1610 is detected and any spaces 1620 adjacent to the newly appearing tooth 1610 are not adjusted. For example, it would be improper to adjust an IPR for newly appearing tooth 1610, particularly since the tooth is still undergoing a great deal of motion as it grows into the lower dental arch.

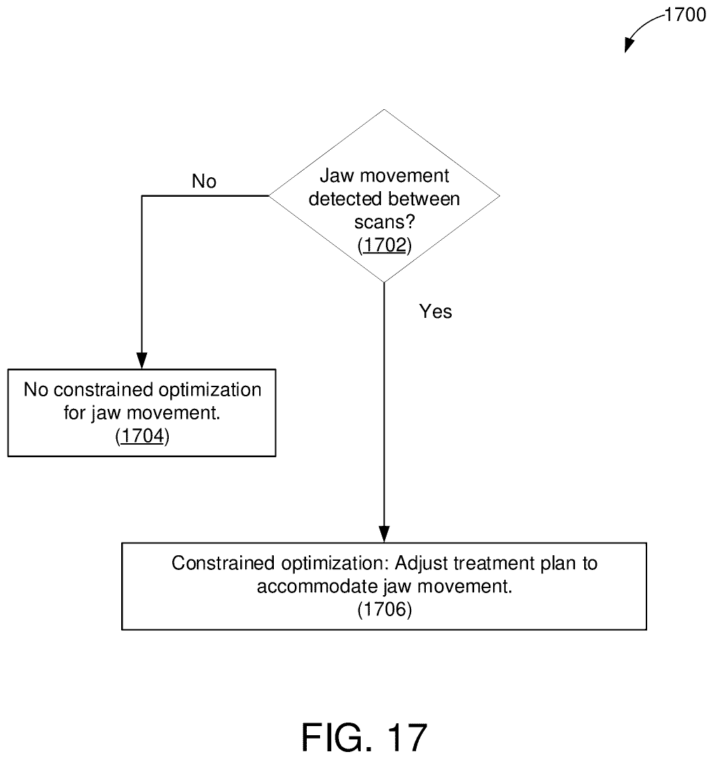

FIG. 17 is a flowchart showing an example method 1700 for accommodating movement in a horizontal (sagittal) plane. In general, the method 1700 may be used to adjust a dental treatment plan based on detected movements in a front-to-back (sagittal) direction with respect to the patient. The method 1700 may be wholly or partially included with the method 1400 of FIG. 14, or in some cases wholly or partially included within block 606 of FIG. 6.

The method 1700 can begin in block 1702 where jaw movement is detected between scans. In some examples, previous matching procedures (for example, as described in block 1402 of FIG. 14) may ease the determination of sagittal jaw or tooth movement.

If no jaw movement is detected, then in block 1704 no constrained optimization to accommodate jaw movement is required, and the method 1700 can end. On the other hand, if jaw movement is detected, then in block 1706 the patient's treatment plan may be adjusted or modified. For example, if movement is determined in the sagittal plane, then the treatment plan may be modified by applying or implementing new constraints for anterior-posterior tooth relationships that can restrict tooth movements in any direction except the front-back direction. In some other examples, tooth movement may be made such that inter-arch collisions and anterior-posterior relationships are dependent on the relative position of jaws based on the same timeline from the patient's original dental treatment plan.

In general, jaw movement is detected as described with respect to block 1702. In some examples, additional conditions can generally be true before constrained optimization is performed for detected jaw movement. A first condition may be that a directive for “maintain options” is not chosen with respect to anterior-posterior relationships. A second condition is that class II/III correction simulation option is selected.

In some examples, features of block 1706 may include copying jaw movements from a special timeline (an object which contains teeth and jaws movements), which contains jaws movements, from a previous dental treatment plan and copy Y-axis translation of tooth movements into the target position of a new, or optimized treatment plan. The optimized treatment plan may allow only horizontal movement along the sagittal plane, considering already made movement during the previous treatment.

In some examples, optimized constraints may be made for the anterior-posterior relationships in the optimized dental plan to be nearly the same as in the target position of a previous dental treatment plan. Alternatively, or in addition, the optimized constraints may restrict all movements in any direction except for translation in Y-direction i.e., front-back jaw translation.

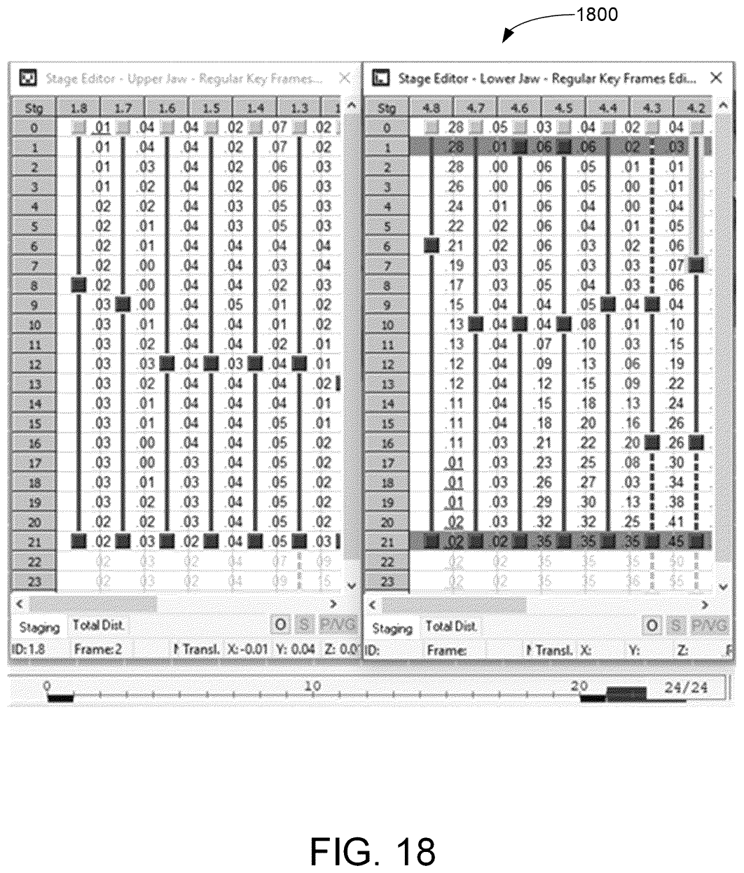

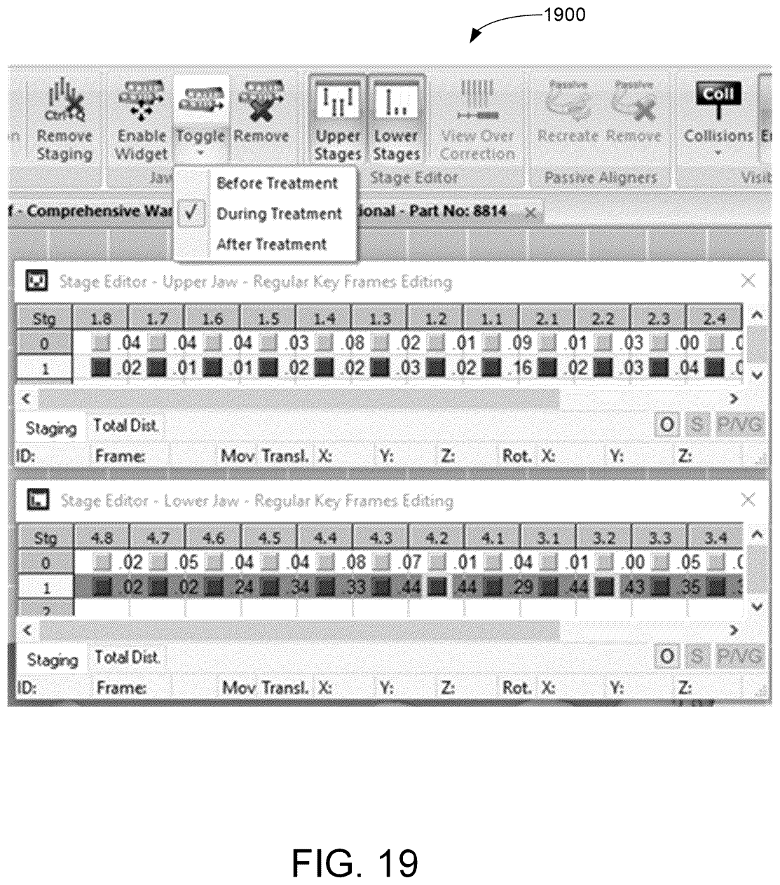

FIG. 18 shows an example user interface 1800 to optimize tooth position while allowing only sagittal plane movement. FIG. 19 shows an example user interface 1900 for lower jaw movement visualization.

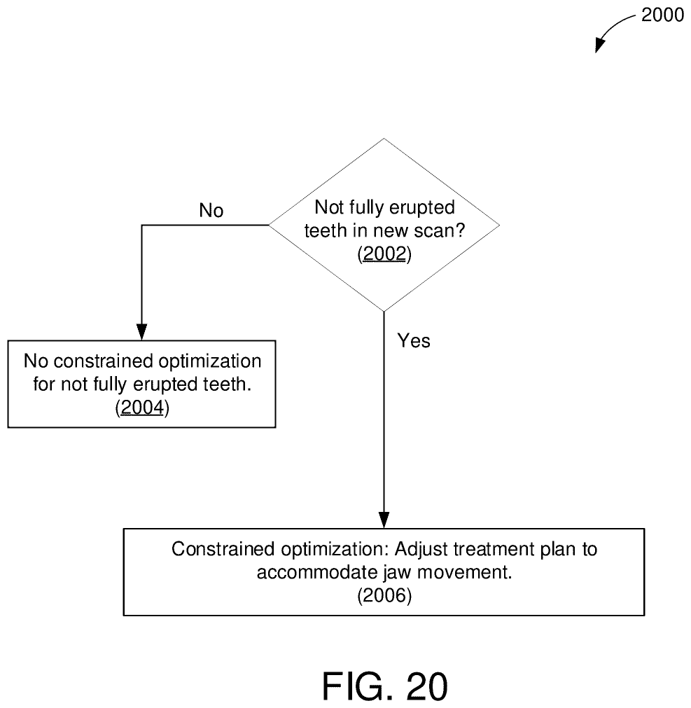

FIG. 20 is a flowchart showing an example method 2000 for accommodating erupting teeth. In general, the method 2000 may be used to adjust a dental treatment plan based on detecting one or more erupting teeth. The method 2000 may be wholly or partially included with the method 1400 of FIG. 14, or in some cases wholly or partially included within block 606 of FIG. 6.

The method 2000 can begin in block 2002 where non-fully erupted teeth are detected in a new dental scan. In some examples, previous matching procedures (for example, as described in block 1402 of FIG. 14) may ease the detection of erupting teeth.

If no non-fully erupting teeth are detected, then in block 2004 no constrained optimization may be performed and the method 2000 may end. On the other hand, if non-fully erupted teeth are detected, then in block 2006 the patient's treatment plan may be adjusted or modified to accommodate the non-fully erupted tooth.

In some examples, constrained optimization may include a restriction of movement for the non-fully erupting teeth. In some other examples, the constrained optimization may include removing collision constraints with neighbor teeth. Thus, movable teeth around not fully erupted teeth will try to preserve space between them as in previous dental treatment plans. In some cases, a tolerance of 0.02 mm for erupting teeth is established to ensure enough free space for the erupting tooth. In addition, the constrained optimization may include a similar logic for primary teeth which have turned into permanent teeth in a dental scan.

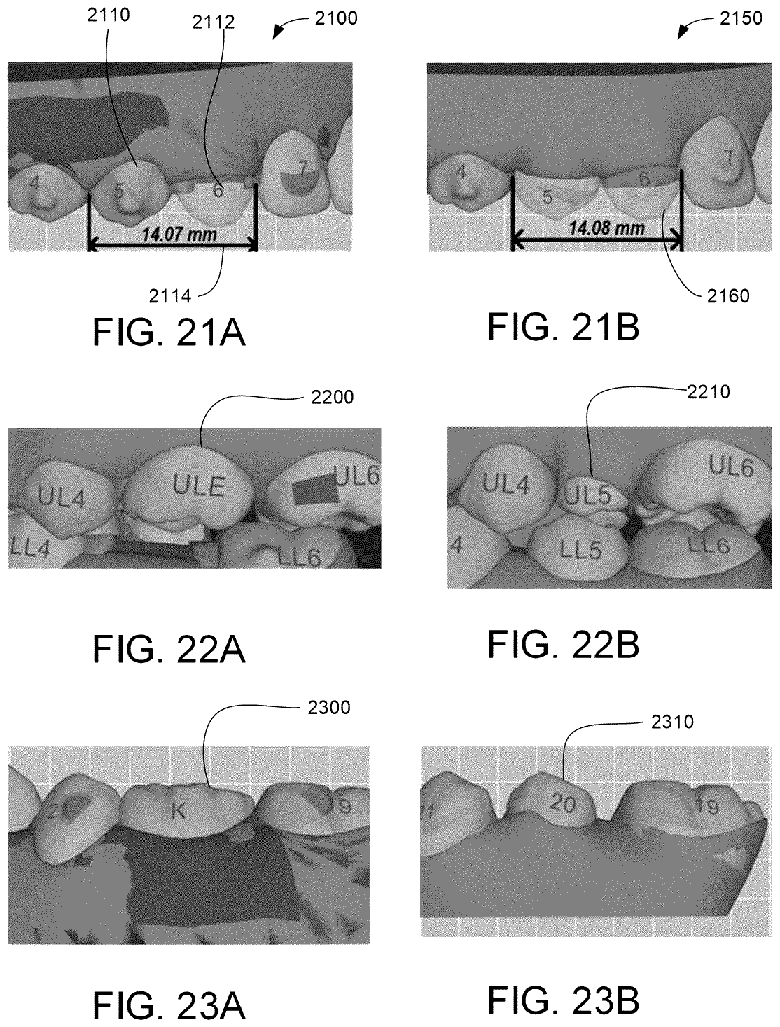

FIG. 21A shows a first view 2100 of a partial dental arch. The partial dental arch may be associated with a subsequent dental scan. Teeth 2110 and 2112 are non-fully erupted teeth. Note that gap 2114 preserves space for the teeth 2110 and 2112 to erupt in to. FIG. 21B shows a second view 2150 of the partial dental arch of FIG. 21A. Tooth 2160 may be a non-fully erupted tooth.

FIG. 22A shows a non-fully erupted tooth 2200 in a current scan (which may be referred to as a subsequent scan). FIG. 22B shows a primary (or deciduous) tooth 2210 that may be associated with an earlier scan. The non-fully erupted tooth 2200 can benefit from additional space being reserved for the tooth 2200 as it grows in to replace tooth 2210.

FIG. 23A shows a permanent tooth 2300 erupting into position in a subsequent scan. FIG. 23B shows a primary tooth 2310 in position during an earlier scan. The permanent tooth 2300 replaces the primary tooth 2310 and benefits from having space reserved for the tooth as it grows.

Notably, there are secondary cases in orthodontics when a doctor sends patient's jaw scans in the middle of a treatment and asks for a treatment to be built that starts from the scan and results in the same final position as the previous treatment. If the teeth from the new order are put into their position from the previous order, the resulting position will likely have clinically inacceptable collisions between teeth because of scanning error or changed teeth shapes.

We solve this problem by running an optimization task on the new order to make collisions clinically acceptable and reduce the amount of manual work by a CAD (Computer Aided Design) designer. The optimization task is formulated in terms of positional characteristics which must be achieved. The goal values of these characteristics are taken from the previous order. Solved optimization task results in a final position of the new order with the values of positional characteristics being the same as in the previous order.

To correctly apply this approach, positional characteristics in both new and previous orders must be evaluated in the same terms. It means that tooth/jaw features which are used to define positional characteristics must describe the same physical parameters on both 3D representations. The methods and apparatuses described herein may address these issues.

To achieve high similarity in tooth/jaw features between new and previous order, the following is done:
Calculate tooth/jaw features only once for the new order
Import tooth/jaw features from the new order into the previous order using tooth matching (rule to transform tooth spatial coordinates in the new order into the coordinates of the previous order)

Use imported tooth/jaw features in previous order to formulate positional goals for the import optimization task.

This may overcome: currently available algorithms of tooth feature calculation are not stable. 3D shapes of the same tooth in different orders slightly differ due to different 3D scanners and CAD designers' operations. But even small difference may lead to the location of the tooth feature being significantly different between new and previous orders. See example of difference in the location of tooth buccal ridge (ridges are any linear tooth elevations, named according to their location).

In some situations, tooth shape in the new order may differ significantly from the shape in the previous order due to physical tooth damage/restoration. In such cases it is impossible to calculate tooth/jaw features in the same way without concept of imported features.

Previous approaches used features which were calculated separately in the new and previous order and had lack of similarity of tooth/jaw features. With the new approach and the high similarity of tooth/jaw features between new and previous orders, it is possible to formulate stricter optimization task and to achieve final position in the new order which is significantly closer to the final position in the previous order.

In some cases, the number of tooth pairs with leveling issues decreased from 20% to 1% (leveling issue is discrepancy between leveling in the final position of the new and previous orders which exceeds 0.3 mm). Leveling describes relative position of a pair of teeth in intrusion/extrusion direction. Calculated as a distance between tip points (the farthest point of the tooth along the long axis that lies on the incisal edge) in millimeters.

The number of tooth pairs with X-misalignment issues decreased from 12.5% to 1.5% (X-misalignment issue is discrepancy between X-misalignment in the final position of the new and previous orders which exceeds 0.4 mm). X-misalignment describes relative position of a pair of teeth in buccal/lingual direction. Calculated as a distance between the closest edges of tooth ridges in millimeters.

Inputs of Import Final Position are: new order represented as cut.adf (file with tooth geometries, without treatment) previous order represented as USTreat.adf (file with tooth geometries and full treatment, including final position, staging, attachments, . . . ).

Ideas of this IDF relies on the result of Matching step. To understand the need of this step and its results, remember, that 3D geometry of teeth is represented as a set of points in 3D space, i.e. a set of vectors of length 3. Despite the new and previous orders describe the same patient and the same teeth, 3D coordinates of tooth points differ between the new and previous order.

In order to be able to repeat the final position from the previous order in the new order, you need to define the correspondence between different 3D representations of the same tooth. This correspondence is called matching, i.e. matching is a rule which allows you to get 3D coordinates of the correspondent tooth points in the previous order given the coordinates in the new order (and vice versa).

Ideas of this IDF are applied in Optimization Task step. The purpose of this step is to create clinically acceptable final position in the new order which is as much similar to the final position in the previous order as much as possible. Currently the following approach is used:

1) Formulate optimization task in terms of positional characteristics which must be achieved in the new order. The values of the positional characteristics are taken from the previous order. E.g. the optimization task may contain statement “leveling of teeth 8-9 must belong to interval [1.0 mm−0.2 mm, 1.0 mm+0.2 mm]”, where 1.0 mm is the leveling of teeth 8-9 in the previous order.

2) Solve optimization task.

3) Apply the results of the optimization task as the final position in the new order.

To correctly define goals for the optimization tasks, i.e. positional characteristics to achieve in the new order, we evaluate those characteristics on the previous order and put the correspondent values as the goals for the new order. Since that it is important that positional characteristics are described in the same terms, i.e. by the same tooth/jaw features.

Imported feature concept works as follows:

1) calculate tooth/jaw features in the new order.

2) import tooth/jaw features into the previous order, using matching.

In some examples, corner cases regarding treatment planning optimization may be detected. These include: newly appeared teeth, jaw movements in a previous order, and not fully erupted teeth in a current or previous order.

Key points: 1) do not move new or not fully erupted teeth, 2) do not introduce constraints involving such teeth to the optimization task solving, 3) keep spaces as in previous order for teeth around not fully erupted ones and around primary teeth turned into permanent in the current order, and 4) Copy lower jaw horizontal movement along the sagittal plane from the previous treatment only if it is requested to correct anterior-posterior relationship in both orders. And place jaw movement visualization (set “Toggle” option) at the same position in the treatment as in the previous order: Before treatment, After treatment, During treatment. So the software makes a clinically acceptable final position for all teeth, except for new, not fully erupted teeth or permanent teeth which were primary before. Such teeth are later manually processed by CAD technician.

The Final Position import algorithm consists of 3 steps: 1) Matching shapes of individual teeth of the previous and the new scan, 2) Applying geometric transforms of position of teeth in the final position (treatment goal) from previous order to the teeth in segmented new scan, and 3) Applying constrained optimization to adjust positions of teeth to account for changes in teeth shapes between the two segmented scans, while preserving the doctor's treatment goal. This step tries to fulfill several constraints and targets.

Sometimes this optimization task produced bad results: inappropriate from clinical point of view teeth positions (movements, occlusion, leveling, etc.). So, we were restricting the cases where it was applied. We did not use it for cases with jaw movements because the engine that we use for optimization tasks had not supported jaw movements when we had been developing our code. We also did not use it for the cases where new teeth appeared in the secondary case or for cases where the primary or secondary case had not fully erupted teeth.

So, the initial version of the optimization task was not applied for several types of corner cases: 1) Newly appeared teeth, 2) Jaw movements in previous order, and 3) Not fully erupted teeth in current or previous order. To allow the optimization task to be used for the cases mentioned above, it was necessary to introduce changes both in 2^{nd} and 3^{rd} steps of the Final Position import algorithm, which will be called STEP 2 and STEP 3 below.

Newly appeared teeth: Changes in STEP 3 only. The optimization task support for cases with newly appeared (FIG. 1) has been switched on via: Disabling newly appeared teeth movements during optimization task solving, and Introducing new optimization pattern rule (let us call it ParametricDistribution), which adjusts spaces only for such teeth pairs which do not contain newly appeared teeth. By default, ParametricDistribution rule introduces constraints which deal with the collisions between neighboring teeth in the arch. By default it requires normal contacts between neighboring teeth, but if there was a space between these teeth in the primary final position, it keeps the space. If there was an IPR (Interproximal Reduction) between these teeth in the primary final position, it checks if the position after STEP 2 is closer to an IPR or to a contact and creates an IPR or a contact accordingly.

Jaw movement in previous order: The optimization task support for cases with jaws movements (only class II/III correction simulation via elastics) in previous order (FIG. 2) is switched on only for cases where it is requested to correct anterior-posterior relationship in both orders, only lower jaw has movements and both jaws need to be treated in current order.

The following must be true in Prescription form (FIG. 3): “Maintain” options are not chosen in Anterior-posterior relationship section. “Class II/III Correction Simulation (Elastics Required)” options is used.

Changes in STEP 2. The idea is to take jaw movements from a special timeline (object which contains teeth and jaws' movements), which contains jaws' movements, from the previous order and copy Y-axis translation of the movement into the final position of the current treatment, allowing only horizontal movement along the sagittal plane, considering already made movement during the previous treatment. And set the “Toggle” tool parameter in the current treatment with the same value as in the previous order: Before treatment, After treatment, During treatment (FIG. 4).

Changes in STEP 3. As for the optimization task itself there were 2 optimizations rules introduced: APSubClassSame, which creates constraints for new the anterior-posterior relationships to be nearly the same as in the final position of the previous order, and DualbiteComponents, which restricts all movements in any direction except for translation in Y-direction i.e., front-back jaw translation.

In addition to jaw movement import it is also necessary to take inter-arch collisions and anterior-posterior relationships values dependent on relative position of jaws from the same timeline from the previous order as for the jaw movement.

Not fully erupted teeth: Movable teeth-fully erupted teeth, which has corresponding teeth in the previous treatment, which are not marked as unmovable, which are not permanent in the current order, but were primary in the previous order, which had movements in the previous order.

Changes in STEP 3 only. The optimization task support for cases with not fully erupted teeth (virtual geometry) via: 1) Forbidding movements of such teeth during optimization and 2) Changing ParametricDistribution optimization rule so that there will be no collision constraints with neighbor teeth introduced for such teeth. So, movable teeth around not fully erupted ones will keep space between them as in previous order with a tolerance of 0.02 mm for erupting teeth to have enough free space. In addition, the same logic was added for primary teeth which turned into permanent in the current order.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method for updating a dental treatment plan, the method comprising:
obtaining a dental treatment plan based on a first dental scan, wherein the dental treatment plan includes a first tooth geometry data and a target position of a patient's teeth;
obtaining a second tooth geometry data from a second dental scan performed subsequent to the first dental scan;
comparing features in the first tooth geometry data to features in the second tooth geometry data to determine teeth in the first dental scan that correspond to teeth in the second dental scan;
determining a geometric transformation to map teeth in the first dental scan that correspond to teeth in the second dental scan;
applying the geometric transformation to the patient's teeth in the target position to determine an updated target position; and
optimizing one or more stages of the dental treatment plan based on updated target position.

2. The method of claim 1, wherein optimizing one or more stages comprises applying a constrained optimization adjusting positions of teeth within the one or more stages accounting for changes in teeth shapes between the first dental scan and the second dental scan.

3. The method of claim 1, wherein optimizing one or more stages comprises:
determining that the second dental scan includes at least one newly appearing tooth; and
modifying one or more stages of the dental treatment plan based at least in part on the at least one newly appearing tooth.

4. The method of claim 1, wherein optimizing one or more stages of the dental treatment plan comprises:
detecting jaw movement between the target position of the patient's teeth and the second dental scan; and
modifying one or more stages of the dental treatment plan to accommodate the jaw movement.

5. The method of claim 1, wherein optimizing one or more stages of the dental treatment plan comprises:
determining that the second dental scan includes at least one erupting tooth; and
modifying one or more stages of the dental treatment plan to accommodate the at least one erupting tooth.

6. The method of claim 5, wherein modifying the one or more stages of the dental treatment plan comprises preserving a gap space between teeth in the target position and a gap space between teeth in the one or more modified stages.

7. The method of claim 1, wherein the second dental scan is provided by an intraoral scanner.

8. The method of claim 1, wherein the second dental scan data includes a three-dimensional (3D) scan of the patient's teeth.

9. The method of claim 1, wherein the target position includes a 3D model of the patient's teeth in the target position.

10. The method of claim 9, wherein the 3D model of the patient's teeth includes the first tooth geometry data.

11. The method of claim 1, further comprising generating design files for dental aligners corresponding to optimized stages of the dental treatment plan.

12. A system comprising:
one or more processors; and
a memory configured to store instructions that, when executed by the one or more processors, cause the system to:
obtain a dental plan based on a first dental scan, wherein the dental treatment plan includes a first tooth geometry data and a target position of a patient's teeth;
obtain a second tooth geometry data from a second dental scan performed subsequent to the first dental scan;
compare features in the first tooth geometry data to features in the second tooth geometry data to determine teeth in the first dental scan that correspond to teeth in the second dental scan;
determine a geometric transformation to map teeth in the first dental scan that correspond to teeth in the second dental scan;
apply the geometric transformation to the patient's teeth in the target position to determine an updated target position; and
optimize one or more stages of the dental treatment plan based on updated target position.

13. The system of claim 12, wherein execution of the instructions to optimize one or more stages causes the system to apply a constrained optimization adjusting positions of teeth within the one or more stages accounting for changes in teeth shapes between the first dental scan and the second dental scan.

14. The system of claim 12, wherein execution of the instructions to optimize one or more stages causes the system to:
determine that the second dental scan includes at least one newly appearing tooth; and
modify one or more stages of the dental treatment plan based at least in part on the at least one newly appearing tooth.

15. The system of claim 14, wherein execution of the instructions to modify one or more stages of the dental treatment plan cause the system to adjust interproximal spaces for teeth in the updated target position based at least in part on the at least one newly appearing tooth.

16. The system of claim 12, wherein execution of the instructions to optimize one or more stages causes the system to:
detect jaw movement between the target position of the patient's teeth and the second dental scan; and
modify one or more stages of the dental treatment plan to accommodate the jaw movement.

17. The system of claim 16, wherein the jaw movement is detected substantially in a sagittal plane of the patient.

18. The system of claim 12, wherein execution of the instructions to optimize one or more stages of the dental treatment plan causes the system to:
determine the second dental scan includes at least one erupting tooth; and
modify one or more stages of the dental treatment plan to accommodate the at least one erupting tooth.

19. The system of claim 18, wherein execution of the instructions to modify the one or more stages of the dental treatment plan causes the system to preserve a gap space between teeth in the target position and a gap space between teeth in the one or more modified stages.

20. The system of claim 12, wherein the second dental scan is provided by an intraoral scanner.

21. The system of claim 12, wherein the second dental scan data includes a three-dimensional (3D) scan of the patient's teeth.

22. The system of claim 12, wherein the target position includes a 3D model of the patient's teeth in the target position.

23. The system of claim 12, wherein execution of the instructions cause the system to generate design files for dental aligners corresponding to optimized stages of the dental treatment plan.

24. A non-transitory computer-readable storage medium comprising instructions that, when executed by one or more processors of a system, cause the system to perform operations comprising:
obtaining a dental treatment plan based on a first dental scan, wherein the dental treatment plan includes a first tooth geometry data and a target position of a patient's teeth;
obtaining a second tooth geometry data from a second dental scan performed subsequent to the first dental scan;
comparing features in the first tooth geometry data to features in the second tooth geometry data to determine teeth in the first dental scan that correspond to teeth in the second dental scan;
determining a geometric transformation to map teeth in the first dental scan that correspond to teeth in the second dental scan;
applying the geometric transformation to the patient's teeth in the target position to determine an updated target position; and
optimizing one or more stages of the dental treatment plan based on updated target position.
